(19) United States
(12) Patent Application Publication
(10) Pub. No.: US 2005/0245972 A1
(43) Pub. Date: Nov. 3, 2005

(54) Apparatus and methods for treating arrhythmia and lowering defibrillation threshold

(75) Inventors: Chike O. Onyekaba, Coon Rapids, MN (US); Timothy G. Laske, Shoreview, MN (US); Rick D. McVenes, Isanti, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/834,473

(22) Filed: Apr. 29, 2004

(51) Int. Cl. A61N 1/39

(52) U.S. Cl. 607/5; 607/3

(57) ABSTRACT

A method for treating cardiac arrhythmia includes infusing an agent, adapted to facilitate treating the arrhythmia, directly into a cardiac vein and delivering a defibrillating shock between two electrodes.

### TECHNICAL FIELD

The present invention generally relates to implantable medical devices, and more particularly relates to treating and preventing arrhythmia, and also to lowering the defibrillation threshold for a patient, using an implantable medical device.

### BACKGROUND

Implantable defibrillation devices known in the art of cardiac rhythm management typically include one or a number of electrical leads coupled to a device. The device is typically implanted in a subcutaneous pocket and the lead(s) extend therefrom via a transvenous route into a patient's heart in order to carry electrical pulses, from the device, for pacing, sensing, and defibrillation. A lead is implanted within the heart so that lead electrodes, coupled to conductors carried within a lead body, are positioned for proper sensing and efficient pacing and defibrillation stimulation. An outer shell or can of the device itself is often used as a defibrillation electrode (‘active can’) in conjunction with one or more defibrillation electrodes carried on a lead body. A shadow area of the electrodes and the implanted position of each electrode are factors determining a threshold of shocking energy required to defibrillate the heart (defibrillation threshold—DFT). One commonly used shocking vector is formed between a right ventricular (RV) defibrillation electrode and a device implanted within a left pectoral region implanted device (RV-can); another further includes a third defibrillation electrode positioned within a superior vena cava (SVC), which is electrically common with the can of the device (RV-SVC+can). It would be desirable to augment defibrillation therapy with a means to reduce DFT's or to maintain acceptable DFT's when the other factors, related to shadow area and shocking vectors, are compromised in order to achieve other objectives.

### BRIEF DESCRIPTION OF THE DRAWINGS

The present invention will hereinafter be described in conjunction with the following drawing figures, wherein like numerals denote like elements, and

FIG. 1 is a schematic view, from a posterior perspective, of a defibrillation device, according to one embodiment of the present invention, coupled to a heart; and

FIGS. 2A-C are a schematic section views, from an anterior perspective, of defibrillation devices, according to alternative embodiments, coupled to a heart.

### DETAILED DESCRIPTION

The following detailed description is merely exemplary in nature and is not intended to limit the invention or the application and uses of the invention.

Embodiments of the present invention are in part based on the present inventors' finding of transmural endomyocardial and epicardial retention of infused Indian ink suspension when delivered into a posterior cardiac vein (PCV) or postero-lateral cardiac vein (PLCV) of the canine left ventricle. The Indian ink is used during cardiac fluid delivery studies as an indicator of a tissue's ability to receive an agent by diffusion. Viewing a large cardiac surface area of about 4.0 cm×1.5 cm×1.2 cm on the posterior lateral aspect of the left ventricle revealed a demonstrable ink presence, and endomyocardial cross sectional segments also revealed the ink presence. Histological sections made from the left ventricle posterior section corroborated the other ink retention findings.

The ink retention findings are beneficial because the left ventricle posterior region presents perhaps the greatest challenge to defibrillation therapy for ventricular tachycardia and ventricular fibrillation patients. The posterior region is a low current/electric field density gradient region during defibrillator energy pulse delivery and may respond better to defibrillation therapy if administered an agent for lowering the defibrillation threshold. Embodiments of the present invention are also based in part on the inventors' observation that a PCV or a PLCV is centrally located to provide convenient access for an elongated device such as a lead or a catheter to the left ventricle. Embodiments of the present invention include delivering either an anti-arrhythmic drug or an agent adapted to lower myocardial tissue resistivity, such as a hypertonic saline solution, through an occluded or non-occluded cardiac vein, i.e. the PCV or the PLCV, to facilitate defibrillation therapy.

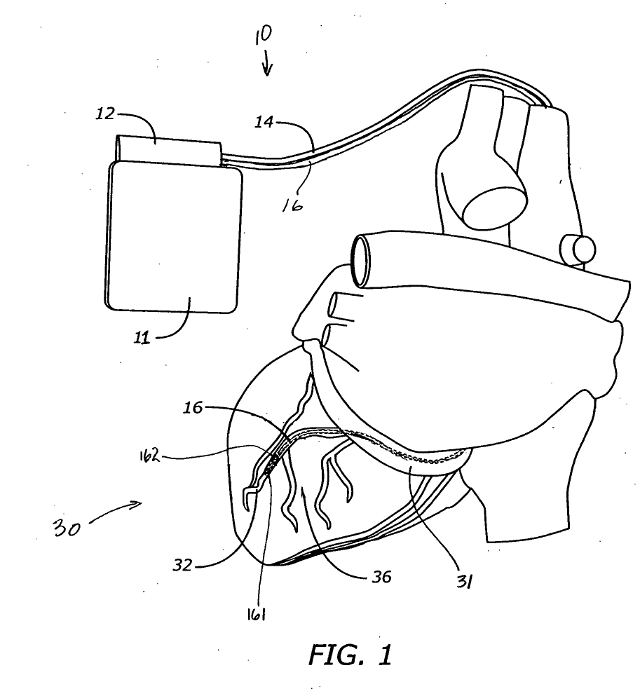

According to one embodiment of the invention, a defibrillation device includes fluid delivery capability along with pacing sensing and defibrillation capability. FIG. 1 is a schematic view, from a posterior perspective, of a defibrillation device 10 coupled to a heart 30 according to one embodiment of the present invention. According to the illustrated embodiment, defibrillation device 10 includes a can 11 enclosing a battery, electronic circuitry and other components adapted to generate electrical pulses for pacing, sensing and defibrillation and a fluid pump; can 11 is coupled to an electrical lead 14 and a fluid delivery catheter 16 via a connector header 12 and may further serve as a defibrillation electrode. The fluid pump may alternately be enclosed in a separate can; according to either embodiment the fluid pump may include a reservoir, which may be refilled via an injection port, and a peristaltic pump, which propels a bolus of infusate out from the reservoir through catheter 16; an example of such a pump is the Medtronic SynchroMed™. The fluid pump may alternately include an osmotic exchange mechanism that self replenishes the reservoir thereby precluding the need for manual trans-dermal reservoir replenishment.

FIG. 1 further illustrates catheter 16 implanted within a PCV 32 along a left ventricular posterior region 36; an infusion port 161 of catheter 16 is positioned within PCV 32, which branches off from a coronary sinus 31, approximately 2 to 3 cm from coronary sinus 31 according to one embodiment. PCV 32 may be occluded prior to infusion by means of a polymeric collar 162 formed about catheter 16 and positioned proximal to infusion port 161; collar 162 can be made from an elastomer capable of swelling upon absorption of moisture or an inflatable balloon known to those skilled in the art. Defibrillation device 10 can also be adapted to infuse anti-arrhythmic fluids to an occluded coronary sinus 31 to treat atrial fibrillation as well.

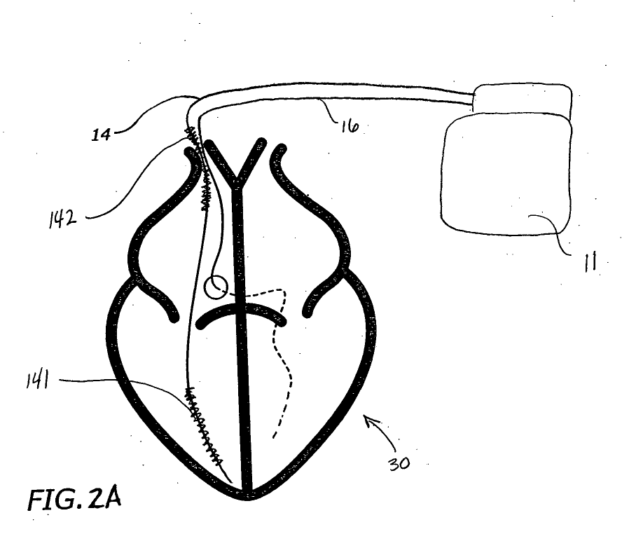

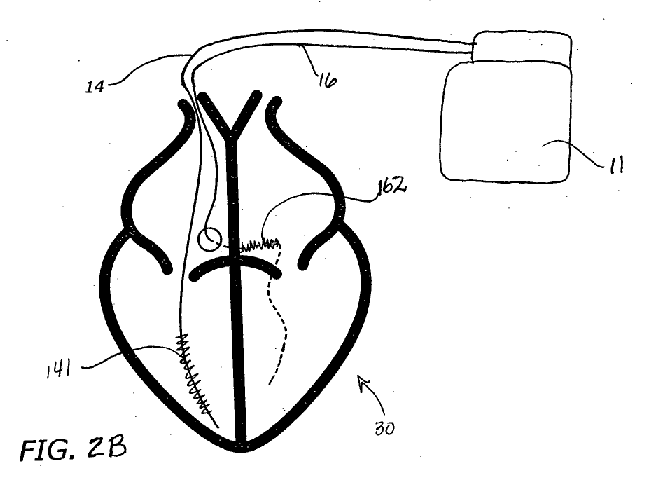

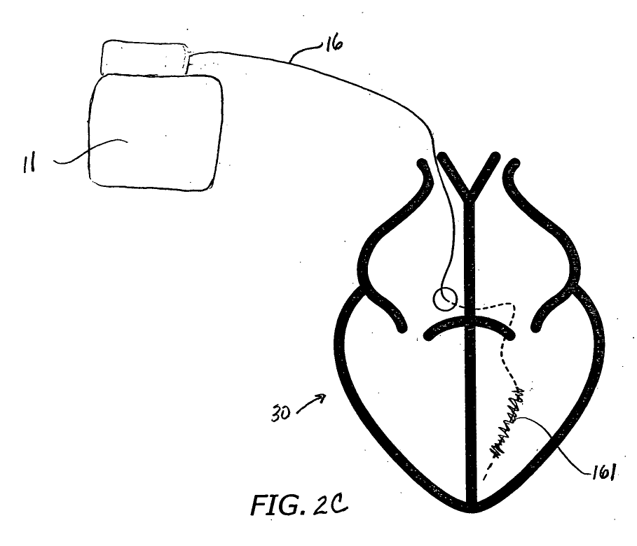

FIGS. 2A-B are schematic section views, from an anterior perspective, wherein an implant site, according to some embodiments, for lead 14 can be seen. FIG. 2A illustrates a defibrillation system including can 11 implanted in a left pectoral region and lead 14 implanted in a right ventricular apex; lead 14 includes a right ventricular electrode 141 (RV electrode) and a superior vena cava defibrillation electrode 142 (SVC electrode); can 11 further serves as another electrode electrically common with SVC electrode 142, according to some embodiments. According to one embodiment, a vector for high voltage defibrillation is formed between RV electrode 141 and a pair of can 11 and SVC electrode 141 (RV-can+SVC); according to alternate embodiments either second defibrillation electrode 142 or can 11 is not included as an electrode in the system (i.e. alternate vectors include RV-SVC and RV-can). FIG. 2B illustrates another embodiment of the present invention wherein SVC electrode 142 is not included and a coronary sinus defibrillation electrode 162 is incorporated on catheter 16, replacing SVC electrode 142 in the aforementioned shocking vectors. FIG. 2C illustrates yet another embodiment of a defibrillation system, wherein catheter 16 includes a cardiac vein defibrillation electrode 161 (CV electrode) intended to form a shocking vector with can 11, which is positioned in a right pectoral region as opposed to the left pectoral region as previously illustrated. It should be noted that CV electrode 161 may be implanted in any of the veins passing along left ventricular posterior region 36 (FIG. 1) according to various embodiments of the present invention.

Although not shown, the defibrillation devices of FIGS. 2A-C further include one or more additional electrodes, which may be included in lead 14 and or catheter 16, for sensing cardiac activity so that the devices may determine when heart 30 is fibrillating. According to the present invention, a DFT for a defibrillating shock is influenced by fluid infusion, for example via catheter 16. According to one method of the present invention, once ventricular fibrillation (VF) detection criteria are met, capacitor charging for a high voltage shock commences and the fluid infusion pump is activated to infuse, via catheter 16, either an anti-arrhythmic agent, or an agent adapted to lower myocardial tissue resistivity or a mixture of the two agents into one or more appropriate cardiac veins, for example PCV 32 illustrated in FIG. 1. Examples of anti-arrhythmic agents include but are not limited to amiodarone, ibutelide and procainamide and an example of an agent lowering myocardial tissue resistivity is an electrolyte-based fluid such as a hypertonic saline solution. After infusion, if VF detection criteria are still met, a high voltage shock is delivered, but if fibrillation has ceased, the shock is aborted. The inventors determined that as little as about 3% NaCl in deionized water infused into a cardiac vein has the potential to substantially reduce a DFT. According to some embodiments, a volume of infused agent is between approximately 5 ml and 40 ml.

A shadow area of defibrillation electrodes, i.e. electrodes 141, 142, 161, 162 and can 11, may be smaller than that of those commonly employed because the system also includes the infusion means, described above, which decreases DFT's. According to some embodiments of the present invention, any or all of electrodes 141, 142, 161 and 162 have a diameter less than approximately 0.08 inch or even less than approximately 0.06 inch.

According to one embodiment, a method further determines whether the fluid reservoir needs replenishing; more particularly, defibrillation devices include a detector that determines whether the fluid level in the reservoir is at or below a predetermined level indicating that the reservoir needs replenishing. If the fluid is at or below the predetermined level, an alarm or other alerting device alerts the patient, physician, or other user.

From the above, it is clear that the present invention in its various embodiments facilitates transmural spread and retention of saline or other anti-arrhythmic agents in localized heart regions. Construction methods and materials, which may be employed to realize the defibrillation devices described herein, are well known to those skilled in the art. Further, the apparatus of the present invention provides flexibility in drug delivery and, when combined with the methods of the present invention, may effectively and quickly treat and terminate atrial or ventricular fibrillation.

While some exemplary embodiments have been presented in the foregoing detailed description, it should be appreciated that a vast number of variations exist and the embodiments described herein are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

1. A cardiac defibrillation device, comprising:
a can enclosing components for generating electrical defibrillation pulses;
a first defibrillation electrode, coupled to the components, and a second defibrillation electrode, coupled to the components, the first electrode and the second electrode forming a shocking vector for cardiac defibrillation;
a reservoir containing an agent comprising an electrolyte;
a fluid pump in fluid communication with the reservoir; and
a fluid delivery catheter flexibly configured to be insertable into a cardiac vein and coupled to the pump to transport the agent from the fluid reservoir to myocardial tissue in proximity to the cardiac vein.

2. The device of claim 1, wherein the can forms the first defibrillation electrode and the second defibrillation electrode is further coupled to the fluid delivery catheter.

3. The device of claim 1, further comprising:
an electrical lead; and
wherein the can forms the first defibrillation electrode; and
the second defibrillation electrode is further coupled to the lead.

4. The device of claim 1, further comprising:
an electrical lead; and
wherein the first defibrillation electrode and the second defibrillation electrode are each further coupled to the lead.

5. The device of claim 1, further comprising:

5. The device of claim 1, further comprising:
an electrical lead; and
the first electrode is further coupled to the catheter; and
the second electrode is further coupled to the lead.

6. The device of claim 2, wherein a diameter of the second defibrillation electrode is less than approximately 0.08 inches.

7. The device of claim 3, wherein a diameter of the second defibrillation electrode is less than approximately 0.08 inches.

8. The device of claim 2, wherein a diameter of the second defibrillation electrode is less than approximately 0.06 inches.

9. The device of claim 3, wherein a diameter of the second defibrillation electrode is less than approximately 0.06 inches.

10. The device of claim 4, wherein a diameter of the first defibrillation electrode and the second defibrillation electrode is less than approximately 0.08 inches.

11. The device of claim 5, wherein a diameter of the first defibrillation electrode and the second defibrillation electrode is less than approximately 0.08 inches.

12. The device of claim 4, wherein a diameter of the first defibrillation electrode and the second defibrillation electrode is less than approximately 0.06 inches.

13. The device of claim 5, wherein a diameter of the first defibrillation electrode and the second defibrillation electrode is less than approximately 0.06 inches.

14. The device of claim 1, wherein the agent further comprises an anti-arrhythmic drug.

15. The device of claim 14, wherein the drug is selected from the group consisting of amiodarone, ibutelide and procainamide.

16. The device of claim 1, wherein the electrolyte comprises NaCl.

17. A method for treating cardiac arrhythmia, comprising the steps of:
detecting cardiac fibrillation;
infusing an agent, adapted to facilitate treating the arrhythmia, directly into a cardiac vein, the cardiac vein passing along a posterior aspect of a left ventricle; and
delivering a defibrillating shock between two electrodes.

18. The method of claim 17, wherein the agent comprises an electrolyte.

19. The method of claim 18, wherein the electrolyte comprises NaCl.

20. The method of claim 17, wherein the agent comprises an anti-arrhythmic drug.

21. The method of claim 20, wherein the drug is selected from the group consisting of amiodarone, ibutelide and procainamide.

22. The method of claim 17, wherein the cardiac vein is a posterior vein.

23. The method of claim 17, wherein the cardiac vein is a posterior lateral vein.

24. The method of claim 17, wherein the cardiac vein is a coronary sinus.

25. The method of claim 17, wherein the cardiac vein branches off from a coronary sinus.

26. The method of claim 25, wherein an infusion site in the cardiac vein is located approximately 2 cm to approximately 3 cm from the coronary sinus.

27. The method of claim 17, wherein a one of the two electrodes is positioned in a right ventricle.

28. The method of claim 17, wherein a one of the two electrodes is positioned in a coronary sinus.

29. The method of claim 17, wherein a one of the two electrodes is positioned in the cardiac vein.

30. The method of claim 17, wherein a volume of the agent infused is between approximately 5 ml and approximately 40 ml.

31. The method of claim 17, further comprising the steps of:
detecting whether the agent is at or below a predetermined level; and
actuating an alerting device in response to the detection.
